(19) United States
(12) Patent Application Publication
(10) Pub. No.: US 2008/0025965 A1
(43) Pub. Date: Jan. 31, 2008

(54) Methods and compositions for the treatment of diseases or conditions associated with increased C-reactive protein levels

(76) Inventor: Jan Lessem, Natick, MA (US)

Correspondence Address: CLARK & ELBING LLP, 101 FEDERAL STREET, BOSTON, MA, 02110, US

(21) Appl. No.: 11/810,544

(22) Filed: Jun. 6, 2007

Related U.S. Application Data
(60) Provisional application No. 60/811,690, filed on Jun. 7, 2006.

(51) Int. Cl. A61K 31/519; A61K 31/55; A61K 31/553; A61K 38/47; A61P 43/00

(52) U.S. Cl. 424/94.61; 514/211.13; 514/214.02; 514/217; 514/262.1

(57) ABSTRACT

The invention features methods and compositions for reducing the serum C-reactive protein (CRP) level in a patient in need thereof, and for treating diseases and conditions associated with an increased serum CRP level. The invention also features methods and compositions for treating a patient diagnosed with, or at risk of developing, periodontal disease by administering a tricyclic compound or an analog thereof and/or a tetra-substituted pyrimidopyrimidine or an analog thereof. The invention also features methods and compositions for reducing the serum CRP level in a patient in need thereof, and for treating diseases and conditions associated with an increased serum CRP level.

### CROSS-REFERENCE TO RELATED APPLICATIONS

This application claims benefit from U.S. Provisional Application No. 60/811,690, filed Jun. 7, 2006, hereby incorporated by reference.

### BACKGROUND OF THE INVENTION

The invention relates to the treatment of diseases and conditions associated with an increased serum C-reactive protein (CRP) level.

CRP is an essential human acute-phase reactant produced in the liver in response to a variety of inflammatory cytokines. The protein is highly conserved and considered to be an early indicator of infectious or inflammatory conditions. Plasma CRP levels increase 1,000-fold in response to infection, ischemia, trauma, burns, and inflammatory conditions. Since the biological half-life of CRP is not influenced by age, liver or kidney function or pharmacotherapy, it is reliable biochemical marker for tissue destruction, necrosis and inflammation and its measurement is widely used to monitor various inflammatory states, angina pectoris, vascular insults, end-stage renal disease, obesity and atherosclerosis.

CRP has recently been shown to be an independent marker for cardiovascular disease. The American Heart Association and Centers for Disease Control and Prevention issued statements recommending CRP be used as a risk marker for cardiovascular disease with a Framingham risk score of between 10% and 20%. Based on their recommendations, CRP levels <1 mg/L are considered low risk, levels from 1 to 3 mg/L represents average risk, and levels>3 mg/L are considered high risk.

Agents that reduce the level of serum CRP would be useful for treating numerous diseases and conditions.

### SUMMARY OF THE INVENTION

In one aspect, the invention features a method for treating periodontal disease (e.g., periodontitis, gingivitis) in a patient by administering (i) a tricyclic compound; and (ii) a tetra-substituted pyrimidopyrimidine or an adenosine activity upregulator in amounts and for a duration that together are sufficient to treat periodontal disease.

In a related aspect, the invention features a method for reducing the serum CRP level in a patient in need thereof by administering to the patient (i) a tricyclic compound; and (ii) a tetra-substituted pyrimidopyrimidine or an adenosine activity upregulator in amounts and for a duration that together are sufficient to reduce the serum CRP level in the patient.

In another related aspect, the invention features a method for treating a disease or condition associated with an increased serum CRP level (e.g., cardiovascular disease, atherosclerosis, hypertension, giant cell arteritis, Kawasaki disease, familial cold urticaria, angina pectoris, vascular insults, end-stage renal disease, colon cancer, lymphoma, sarcoma, pancreatitis, or pancreatic cancer) in a patient in need thereof by administering to the patient (i) a corticosteroid; and (ii) a tetra-substituted pyrimidopyrimidine or an adenosine activity upregulator, wherein the two drugs are administered in amounts and for a duration that together are sufficient to reduce the serum CRP level in the patient.

In any of the foregoing aspects, the two drugs can be formulated in a single pharmaceutical composition, or can be in separate formulations and administered simultaneously (i.e., within an hour of each other), within 2, 4, 6, 8, 12, or 16 hours of each other, or within 1, 5, 7, 10, or 14 days of each other.

In any of the above aspects, a third agent may also be administered to the patient. Suitable agents include antibiotics (penicillin, cephalosporin, tetracycline, oxytetracycline, chlortetracycline, metronidazole, chloramphenicol, streptomycin, neomycin, sulfonamides, phenolic compounds, quarternary ammonium compounds, minocycline, doxycycline); antiseptics (e.g., chlorhexidine); NSAIDs (e.g., flurbiprofen, carprofen, diclofenac, fenbufen, fenclozic acid, fenoprofen, flufenamic acid, ibuprofen, indomethacin, indoprofen, ketoprofen, lonazolac, loxoprofen, meclofenamic acid, mefanamic acid, naproxen, proprionic acids, salicylic acids, sulindac, tolmetin, meloxicam, oxicams, piroxicam, tenoxicam, etodolac, and oxaprozin); tranexamic acid, allantoin; epsilon-aminocaproic acid; lysozyme; dihydrocholesterol; beta-glycyrrhetinic acid; platelet aggregation inhibitors (e.g., abciximab, aspirin, cilostazol, clopidogrel, eptifibatide, ticlopidine, or tirofiban); anticoagulants (e.g., dalteparin, danaparoid, enoxaparin, heparin, tinzaparin, or warfarin); antipyretics (e.g., acetaminophen); ticlopidine; clopidogrel; angiotensin converting enzyme inhibitors; beta blockers; pentoxifylline; cilostazol; estrogen replacement therapy; and lipid-lowering agents (e.g., cholestyramine, colestipol, nicotinic acid, gemfibrozil, probucol, ezetimibe, or statins such as atorvastatin, rosuvastatin, lovastatin simvastatin, pravastatin, cerivastatin, and fluvastatin). These secondary therapeutic agents may be administered within 14 days, 7 days, 1 day, or 12 hours of administration of a tricyclic compound and/or a tetra-substituted pyrimidopyrimidine, or simultaneously therewith. The additional therapeutic agents may be present in the same or different pharmaceutical compositions as the tricyclic compound and/or tetra-substituted pyrimidopyrimidine of the invention. When present in different pharmaceutical compositions, different routes of administration may be used.

In other embodiments, the tricyclic compound and the tetra-substituted pyrimidopyrimidine are the only two active ingredients (although excipients will generally also be present).

The invention also features a device for administering drugs to the periodontal pockets of a patient having periodontal disease. The device includes a tricyclic compound and a tetra-substituted pyrimidopyrimidine or an adenosine activity upregulator capable of being released into the periodontal pockets of the patient in periodontal disease-treating amounts. Additional drugs, such as those listed above, can also be included in this device.

The invention also features a variety of kits. One kit includes (i) a tricyclic compound; (ii) a tetra-substituted pyrimidopyrimidine or an adenosine activity upregulator; and (iii) instructions for administering drugs to a patient having or at risk of having periodontal disease, a patient having an increased serum CRP level, or a patient having or at risk of having periodontal disease. In one embodiment, the two drugs are contained within a single composition.

Another kit of the invention includes either a tricyclic compound or a tetra-substituted pyrimidopyrimidine or an adenosine activity upregulator and instructions for administering both a tricyclic compound and a tetra-substituted pyrimidopyrimidine or an adenosine activity upregulator to a patient having periodontal disease, a patient having an increased serum CRP level, or a patient having or at risk of having periodontal disease.

In certain embodiments of any of the foregoing aspects of the invention, the tricyclic compound is selected from the group consisting of amitriptyline, amoxapine, clomipramine, dothiepin, doxepin, desipramine, imipramine, lofepramine, loxapine, maprotiline, mianserin, mirtazapine, oxaprotiline, nortriptyline, octriptyline, protriptyline, and trimipramine, and the tetra-substituted pyrimidopyrimidine is dipyridamole. In one particular embodiment, the tricyclic compound is amoxapine and the tetra-substituted pyrimidopyrimidine is dipyridamole.

While the invention is described generally in terms of combination therapy, it is understood that either agent (i.e., the tricyclic compound or the tetra-substituted pyrimidopyrimidine or an adenosine activity upregulator) can be used as a monotherapy to treat periodontal disease or reduce the serum CRP level in a patient needing such treatment. Thus, any of the foregoing methods can be performed (and any of the foregoing devices produced) employing only a tricyclic compound or only a tetra-substituted pyrimidopyrimidine. As an example, in one embodiment, the invention features a method of treating periodontal disease by administering dipyridamole as a monotherapy to treat periodontal disease or reduce the serum CRP level.

Compounds useful in the invention include those described herein in any of their pharmaceutically acceptable salts or other forms, including isomers such as diastereomers and enantiomers, esters, solvates, and polymorphs thereof, as well as racemic mixtures and pure isomers of the compounds described herein. As an example, by “loxapine” is meant the free base, as well as any pharmaceutically acceptable salt thereof (e.g., loxapine hydrochloride, loxapine succinate).

By “tricyclic compound” is meant a compound having one the formulas (I), (II), (III), or (IV): 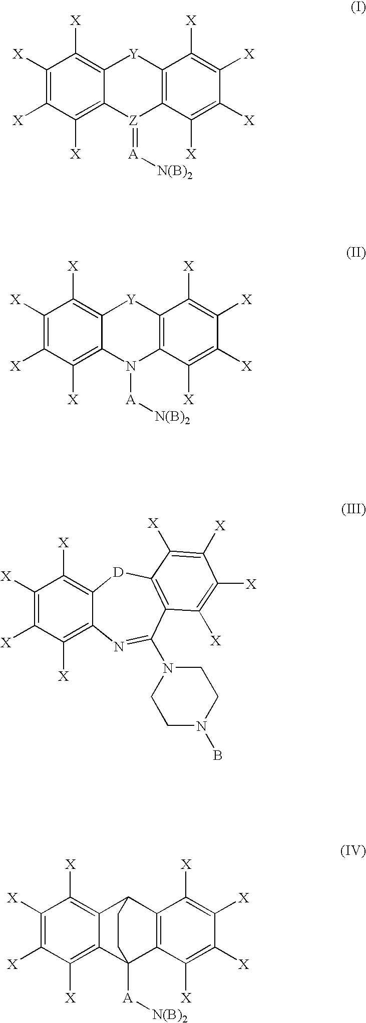
wherein each X is, independently, H, Cl, F, Br, I, CH₃, CF₃, OH, OCH₃, CH₂CH₃, or OCH₂CH₃; Y is CH₂, O, NH, S(O)₀₋₂, (CH₂)₃, (CH)₂, CH₂O, CH₂NH, CHN, or CH₂S; Z is C or S; A is a branched or unbranched, saturated or monounsaturated hydrocarbon chain having between 3 and 6 carbons, inclusive; each B is, independently, H, Cl, F, Br, I, CX₃, CH₂CH₃, OCX₃, or OCX₂CX₃; and D is CH₂, O, NH, or S(O)₀₋₂. In preferred embodiments, each X is, independently, H, Cl, or F; Y is (CH₂)₂, Z is C; A is (CH₂)₃; and each B is, independently, H, Cl, or F. Other tricyclic compounds are described below. Tricyclic compounds include tricyclic antidepressants such as amoxapine, 8-hydroxyamoxapine, 7-hydroxyamoxapine, loxapine (e.g., loxapine succinate, loxapine hydrochloride), 8-hydroxyloxapine, amitriptyline, clomipramine, doxepin, imipramine, trimipramine, desipramine, nortriptyline, and protriptyline, although compounds need not have antidepressant activities to be considered tricyclic compounds of the invention.

By “tetra-substituted pyrimidopyrimidine” is meant a compound of formula (V): 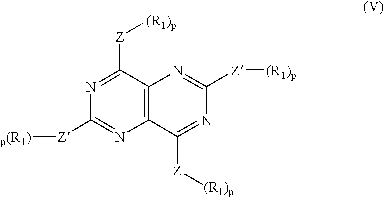
wherein each Z and each Z′ is, independently, N, O, C,

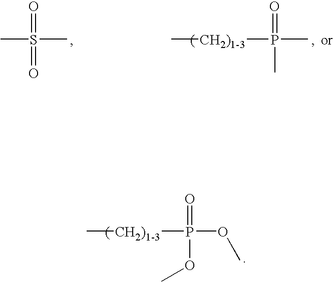

When Z or Z′ is O or 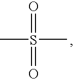
then p=1, when Z or Z′ is N, 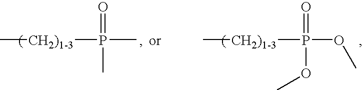
then p=2, and when Z or Z′ is C, then p=3. In formula (V), each R₁ is, independently, X, OH, N-alkyl (wherein the alkyl group has 1 to 20, more preferably 1-5, carbon atoms); a branched or unbranched alkyl group having 1 to 20, more preferably 1-5, carbon atoms; or a heterocycle, preferably as defined in formula (Y), below. Alternatively, when p>1, two R₁ groups from a common Z or Z′ atom, in combination with each other, may represent —(CY₂)ₖ— in which k is an integer between 4 and 6, inclusive. Each X is, independently, Y, CY₃, C(CY₃)₃, CY₂CY₃, (CY₂)₁₋₅OY, substituted or unsubstituted cycloalkane of the structure CₙY₂ₙ₋₁, wherein n=3-7, inclusive. Each Y is, independently, H, F, Cl, Br, or I. In one embodiment, each Z is the same moiety, each Z′ is the same moiety, and Z and Z′ are different moieties.

Particularly useful tetra-substituted pyrimidopyrimidines for use in the methods, kits, and compositions of the invention are dipyridamole (also known as 2,6-bis(diethanolamino)-4,8-dipiperidinopyrimido(5,4-d)pyrimidine); 2,6-disubstituted 4,8-dibenzylaminopyrimido[5,4-d]pyrimidines; mopidamole; dipyridamole monoacetate; R-E 244 (1-((2,7-bis(2-methyl-4-morpholinyl)-6-phenyl-4-pteridinyl)(2-hydroxyethyl)amino)-2-propanol); TX-3301 (asasantin); NU3026 (2,6-di-(2,2-dimethyl-1,3-dioxolan-4-yl)-methoxy-4,8-di-piperidinopyrimidopyrimidine); NU3059 (2,6-bis-(2,3-dimethyoxypropoxy)-4,8-di-piperidinopyrimidopyrimidine); NU3060 (2,6-bis[N,N-di(2-methoxy)ethyl]-4,6-di-piperidinopyrimidopyrimidine); and NU3076 (2,6-bis(diethanolamino)-4,8-di-4-methoxybenzylaminopyrimidopyrimidine). Other tetra-substituted pyrimidopyrimidines are described in U.S. Pat. Nos. 3,031,450 and 4,963,541, hereby incorporated by reference.

By “adenosine activity upregulator” is meant adenosine and any compounds that mimic or potentiate the physiological effects of adenosine, such as adenosine receptor agonists, adenosine transport inhibitors, adenosine kinase inhibitors, and phosphodiesterase (PDE) inhibitors, as described herein.

By a “low dosage” is meant at least 5% less (e.g., at least 10%, 20%, 50%, 80%, 90%, or even 95%) than the lowest standard recommended dosage of a particular compound formulated for a given route of administration for treatment of any human disease or condition. For example, a low dosage of tetra-substituted pyrimidopyrimidine formulated for administration by inhalation will differ from a low dosage of tetra-substituted pyrimidopyrimidine formulated for oral administration.

By a “high dosage” is meant at least 5% (e.g., at least 10%, 20%, 50%, 100%, 200%, or even 300%) more than the highest standard recommended dosage of a particular compound for treatment of any human disease or condition.

By a “moderate dosage” is meant the dosage between the low dosage and the high dosage.

By “treating” is meant administering or prescribing a pharmaceutical composition for the treatment or prevention of a disease or condition.

By “patient” is meant any animal (e.g., a human). Other animals that can be treated using the methods, compositions, and kits of the invention include horses, dogs, cats, pigs, goats, rabbits, hamsters, monkeys, guinea pigs, rats, mice, lizards, snakes, sheep, cattle, fish, and birds. In one embodiment of the invention, the patient subject to a treatment described herein does not have clinical depression, an anxiety or panic disorder, an obsessive/compulsive disorder, alcoholism, an eating disorder, an attention-deficit disorder, a borderline personality disorder, a sleep disorder, a headache, premenstrual syndrome, an irregular heartbeat, schizophrenia, Tourette's syndrome, or phobias.

By “an amount sufficient” is meant the amount of a compound, in a combination of the invention, required to treat or prevent a disease or condition in a clinically relevant manner. A sufficient amount of an active compound used to practice the present invention for therapeutic treatment of particular diseases and conditions caused varies depending upon the manner of administration, the age, body weight, and general health of the patient. Ultimately, the prescribers will decide the appropriate amount and dosage regimen.

By “more effective” is meant that a method, composition, or kit exhibits greater efficacy, is less toxic, safer, more convenient, better tolerated, or less expensive, or provides more treatment satisfaction than another method, composition, or kit with which it is being compared. Efficacy may be measured by a skilled practitioner using any standard method that is appropriate for a given indication.

The term “periodontal disease” encompasses a variety of conditions, including gingivitis and periodontitis, as well as diseases of tissues that surround and support teeth, including the gingiva, cementum, periodontal ligament, alveolar process bone, and dental supporting bone.

By “a disease or condition associated with an increased serum CRP level” is meant any disease or disorder in which the level of serum CRP may be elevated compared to normal controls. Typically a serum CRP level of >3 mg/L is considered elevated. Such diseases and conditions associated with an increased serum CRP level include cardiovascular disease, atherosclerosis, hypertension, giant cell arteritis, Kawasaki disease, familial cold urticaria, angina pectoris, vascular insults, end-stage renal disease, colon cancer, lymphoma, sarcoma, pancreatitis, and pancreatic cancer.

By “sustained release” or “controlled release” is meant that the therapeutically active component is released from the formulation at a controlled rate such that therapeutically beneficial blood levels (but below toxic levels) of the component are maintained over an extended period of time ranging from e.g., about 12 to about 24 hours, thus, providing, for example, a 12 hour or a 24 hour dosage form.

The term “pharmaceutically acceptable salt” represents those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. The salts can be prepared in situ during the final isolation and purification of the compounds of the invention, or separately by reacting the free base function with a suitable organic acid. Representative acid addition salts include acetate, adipate, alginate, ascorbate, aspartate, benzenesulfonate, benzoate, bisulfate, borate, butyrate, camphorate, camphersulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, isethionate, lactobionate, lactate, laurate, lauryl sulfate, malate, maleate, malonate, mesylate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like.

Compounds useful in the invention may also be isotopically labeled compounds. Useful isotopes include hydrogen, carbon, nitrogen, oxygen, phosphorous, fluorine, and chlorine, (e.g., ²H, ³H, ¹³C, ¹⁴C, ¹⁵N, ¹⁸O, ¹⁷O, ³¹P, ³²P, ³⁵S, ¹⁸F, and ³⁶Cl). Isotopically-labeled compounds can be prepared by synthesizing a compound using a readily available isotopically-labeled reagent in place of a non-isotopically-labeled reagent.

In the generic descriptions of compounds of this invention, the number of atoms of a particular type in a substituent group is generally given as a range, e.g., an alkyl group containing from 1 to 7 carbon atoms or C₁₋₇ alkyl. Reference to such a range is intended to include specific references to groups having each of the integer number of atoms within the specified range. For example, an alkyl group from 1 to 7 carbon atoms includes each of C₁, C₂, C₃, C₄, C₅, C₆, and C₇. A C₁₋₇ heteroalkyl, for example, includes from 1 to 7 carbon atoms in addition to one or more heteroatoms. Other numbers of atoms and other types of atoms may be indicated in a similar manner.

Other features and advantages of the invention will be apparent from the following detailed description, and from the claims.

### DETAILED DESCRIPTION

The invention features methods and compositions for reducing the serum CRP level in a patient in need thereof, and for treating diseases and conditions associated with an increased serum CRP level. The invention also features methods and compositions for treating a patient diagnosed with, or at risk of developing, periodontal disease by administering a tricyclic compound or an analog thereof and/or a tetra-substituted pyrimidopyrimidine or an analog thereof (e.g., an adenosine activity upregulator) to the patient. In one embodiment of the invention, treatment is performed by administering a tricyclic compound and dipyridamole to a patient in need of such treatment.

The invention is described in greater detail below.

Tetra-Substituted Pyrimidopyrimidines

Tetra-substituted pyrimidopyrimidines that are useful in the methods, compositions, and kits of this invention include 2,6-disubstituted 4,8-dibenzylaminopyrimido[5,4-d]pyrimidines. Particularly useful tetra-substituted pyrimidopyrimidines include dipyridamole (also known as 2,6-bis(diethanolamino)-4,8-dipiperidinopyrimido(5,4-d)pyrimidine); mopidamole; dipyridamole monoacetate; R-E 244 (1-((2,7-bis(2-methyl-4-morpholinyl)-6-phenyl-4-pteridinyl)(2-hydroxyethyl)amino)-2-propanol); TX-3301 (asasantin); NU3026 (2,6-di-(2,2-dimethyl-1,3-dioxolan-4-yl)-methoxy-4,8-di-piperidinopyrimidopyrimidine); NU3059 (2,6-bis-(2,3-dimethyoxypropoxy)-4,8-di-piperidinopyrimidopyrimidine); NU3060 (2,6-bis[N,N-di(2-methoxy)ethyl]-4,6-di-piperidinopyrimidopyrimidine); and NU3076 (2,6-bis(diethanolamino)-4,8-di-4-methoxybenzylaminopyrimidopyrimidine). Other tetra-substituted pyrimidopyrimidines are described in U.S. Pat. Nos. 3,031,450 and 4,963,541.

The standard recommended dosage for dipyridamole is 300-400 mg/day.

Adenosine and Adenosine Activity Upregulators

Dipyridamole is an adenosine activity upregulator. If desired, another adenosine activity upregulator can be used in place of dipyridamole in the methods, compositions, and kits of the invention. Suitable adenosine activity upregulators are adenosine receptor agonists, adenosine transport inhibitors, adenosine kinase inhibitors, and phosphodiesterase (PDE) inhibitors, discussed below.

Adenosine Receptor Agonists

Examples of adenosine receptor agonists that can be employed in the methods, compositions, and kits of the invention are adenosine hemisulfate salt, adenosine amine congener solid, N⁶-(4-amino-3-iodophenyl)methyl-5′-N-methylcarboxamidoadenosine (I-AB-MECA); N-((2-methylphenyl)methyl)adenosine (Metrifudil); 2-(1-hexynyl)-N-methyladenosine (HEMADO); N-(1-methyl-2-phenylethyl)adenosine (R-PIA); N⁶—(R-4-hydroxyphenylisopropyl)adenosine (HPIA); N⁶-cyclopentyladenosine (CPA); N⁶-cyclopentyl-2-(3-phenylaminocarbonyltriazene-1-yl)adenosine (TCPA); N-((1S, trans)-2-hydroxycyclopentyl)adenosine (GR 79236); N⁶-cyclohexyladenosine (CHA); 2-chloro-N⁶-cyclopentyladenosine (CCPA); N-ethylcarboxamidoadenosine (NECA); 2-(4-(2-carboxyethyl)phenethylamino)-5′-N-ethylcarboxamidoadenosine (CGS 21680); N⁶-(3-iodobenzyl)-5′-N-methylcarboxamidoadenosine (IB-MECA); 2-(cyclohexylmethylidene hydrazino)adenosine (WRC 0470); 2-(4-(2-carboxyethyl)phenethylamino)-5′-N-ethylcarboxamidoadenosine (CGS 21680); N⁶-(2-(3,5-dimethoxyphenyl)-2-(2-methylphenyl)ethyl)adenosine (DPMA); hexynyladenosine-5′-N-ethylcarboxamide (HE-NECA); 2-[(2-aminoethyl-aminocarbonylethyl) phenylethylamino]-5′-N-ethyl-carboxamidoadenosine (APEC); 2-chloro-N⁶-(3-iodobenzyl)-5′-N-methylcarboxamidoadenosine (2-Cl-IB-MECA); 2-phenylaminoadenosine (CV 1808); 3′-Aminoadenosine-5′-uronamides; CV Therapeutics™ small molecule drugs Tecadenoson (CVT-510); Regadenoson (CVT 3146); and Carisa (CVT 3033); and Aderis Pharmaceuticals™ small drug molecules 2-[2-(4-chlorophenyl)ethoxy]adenosine (MRE 0094), 1-deoxy-1-[6-[[(iodophenyl)methyl]amino]-9H-purine-9-yl]-N-methyl-(-D-ribofuranuronamide) (CF101), Selodenoson (DTI-0009) and Binodenoson (MRE-0470). Other adenosine receptor agonists are those described or claimed in Gao et al., JPET, 298: 209-218 (2001); U.S. Pat. Nos. 5,278,150, 5,877,180, 6,232,297; U.S. Patent Application Publication No. 20050261236, and PCT Publication No. WO/9808855, incorporated herein by reference.

Adenosine Transport Inhibitors

Adenosine transport inhibitors that can be employed in the methods, compositions, and kits of the invention include 3-[1-(6,7-diethoxy-2-morpholinoquinazolin-4-yl)piperidin-4-yl]-1,6-dimethyl-2,4(1H,3H)-quinazolinedione hydrochloride (KF24345); 6-(4-nitrobenzyl)-thioinosine (NBI) and 6-(2-hydroxy-5-nitrobenzyl)-thioguanosine (NBG); 6-[4-(1-cyclohexyl-1H-tetrazol-5-yl)butoxy]-3,4-dihydro-2(1H)-quinolinone (Cilostazol); (2-amino-4,5-dimethyl-3-thienyl)-[3-(trifluoromethyl)phenyl]methanone (PD 81723); 3,7-dihydro-3-methyl-1-(5-oxohexyl)-7-propyl-1H-purine-2,6-dione (propentofylline); 6-[(4-nitrobenzyl)thio]-9-β-D-ribofuranosylpurine (nitrobenzylthioinosine) (NBMR); 3,4,5-trimethoxy-, (tetrahydro-1H-1,4-diazepine-1,4(5H)-diyl)di-3,1-propanediyl benzoic acid, ester (dilazep); hexobendine; dipyridamole; and adenosine transport inhibitors described in Fredholm, J. Neurochem. 62:563-573 (1994), Noji et al., J. Pharmacol. Exp. Ther. 300:200-205 (2002); and Crawley et al.; Neurosci. Lett. 36:169-174 (1983), each of which is incorporated herein by reference.

Adenosine Kinase Inhibitors

Adenosine kinase inhibitors are adenosine activity upregulators that can be used in the methods, compositions, and kits of the invention. Adenosine kinase inhibitors are generally described as either nucleoside-like, or nonnucleoside-like.

Nucleoside-Like Adenosine Kinase Inhibitors

Nucleoside-like adenosine kinase inhibitors that can be used in the methods, compositions, and kits of the invention include 5-iodotubercidin (5IT) and 2-diaryltubercidin analogues; 5′-deoxo-5′-deoxy-5-iodotubercidin (5′d-5IT); and 5′-deoxo-5′-aminoadenosine (NH₂dADO). Other nucleoside-like adenosine kinase inhibitors are described in McGaraughty et al., Current Topics in Medicinal Chemistry 5:43-58 (2005); Ugarkar, J. Med. Chem. 43:2883-2893 (2000); Ugarkar et al., J. Med. Chem. 43:2894-2905 (2000); Kaplan and Coyle, Eur. J. Pharmacol. 1:1-8 (1998); and Sinclair et al. Br. J. Pharmacol. 5:1037-1044 (2001), each of which is incorporated herein by reference.

Nonnucleoside-Like Adenosine Kinase Inhibitors

Nonnucleoside-like adenosine kinase inhibitors that can be used in the methods, compositions, and kits of the invention include 5-bromopyrrolopyrrolidine; 4-amino-5-(3-bromophenyl)-7-(6-morpholino-pyridin-3-yl)pyrido[2,3-d]pyrimidine (ABT-702). Other nonnucleoside-like AK inhibitors are described in McGaraughty et al., Current Topics in Medicinal Chemistry 5:43-58 (2005), Gomtsyan and Lee, Current Pharmaceutical Design 10:1093-1103 (2004); Jarvis et al. J. Pharm. Exp. Ther. 295:1156-1164 (2000); Kowaluk, et al. J. Pharm. Exp. Ther. 295:1165-1174 (2000); and German Patent Application DE 10141212 A1, each of which is incorporated herein by reference.

Phosphodiesterase Inhibitors

Several isozymes of phosphodiesterases act as regulatory switches by catalyzing the degradation of cAMP to adenosine-5-monophosphate (5′-AMP). Inhibitors of phosphodiesterases can lead to an increase in cAMP levels, which in turn can lead to an increase in antiinflammatory actions.

Type I Phosphodiesterase Inhibitors

Type I PDE inhibitors that can be employed in the methods, compositions, and kits of the invention include (3-alpha, 16-alpha)-eburnamenine-14-carboxylic acid ethyl ester (Vinpocetine); 1 8-methoxymethyl-3-isobutyl-1-methylxantine (MIMX); 1-carboxy-2,3,4,4a,4b,5,6,6a,6b,7,8,8a,8b,9,10,10a,14,16,17,17a,17b,18,19,19a,19b, 20,21,21a,21b,22,23,23a-dotriacontahydro-14-hydroxy-8a,10a-bis(hydroxymethyl)-14-(3-methoxy-3-oxopropyl)-1,4,4a,6,6a,17b,19b,21b-octamethyl beta-D-glucopyranosiduronic acid (Ks-505a); cis-5,6a,7,8,9,9a-hexahydro-2-(4-(trifluoromethyl)phenylmethyl)-5-methyl-cyclopent (4,5)imidazo[2,1-b]purin-4(3H)-one (SCH 51866); and 2-o-propoxyphenyl-8-azapurine-6-one (Zaprinast). Other Type I PDE inhibitors are described in U.S. Patent Application Nos. 20040259792 and 20050075795, incorporated herein by reference.

Type II Phosphodiesterase Inhibitors

Type II PDE inhibitors that can be employed in the methods, compositions, and kits of the invention include erythro-9-(2-hydroxy-3-nonyl)adenine (EHNA); 2,3,6,7-tetrahydro-9,10-dimethoxy-3-methyl-2-((2,4,6-trimethylphenyl)imino)-4H-pyrimido(6,1-a)isoquinolin-4-one (trequinsin); ND7001 (Neuro3D Pharmaceuticals); and BAY 60-7550 (Alexis Biochemicals). Other Type II PDE inhibitors are described in U.S. Patent Application No. 20030176316, incorporated herein by reference.

Type III Phosphodiesterase Inhibitors

Type III PDE inhibitors that can be employed in the methods, compositions, and kits of the invention include 3-isobutyl-1-methylxanthine (IBMX); 6-dihydro-2-methyl-6-oxo-3,4′-bipyridine)-5-carbonitrile (milrinone); and N-cyclohexyl-4-((1,2-dihydro-2-oxo-6-quinolinyl)oxy)-N-methyl-butanamide (cilostamide). Other Type III PDE inhibitors are described in the following patents and patent applications: EP 0 653 426, EP 0 294 647, EP 0 357 788, EP 0 220 044, EP 0 326 307, EP 0 207 500, EP 0 406 958, EP 0 150 937, EP 0 075 463, EP 0 272 914, and EP 0 112 987, U.S. Pat. Nos. 4,963,561; 5,141,931, 6,897,229, and 6,156,753; U.S. Patent Application Nos. 20030158133, 20040097593, 20060030611, and 20060025463; WO 96/15117; DE 2825048; DE 2727481; DE 2847621; DE 3044568; DE 2837161; and DE 3021792, each of which is incorporated herein by reference.

Type IV Phosphodiesterase Inhibitors

Type IV PDE inhibitors that can be employed in the methods, compositions, and kits of the invention include 4-(3-cyclopentyloxy-4-methoxyphenyl)-2-pyrrolidone (rolipram) and 4-(3-butoxy-4-methoxybenzyl)-2-imidazolidinone (Ro20-1724). Other Type IV PDE inhibitors are described in the following patents, patent applications, and references: U.S. Pat. Nos. 3,892,777, 4,193,926, 4,655,074, 4,965,271, 5,096,906, 5,124,455, 5,272,153, 6,569,890, 6,953,853, 6,933,296, 6,919,353, 6,953,810, 6,949,573, 6,909,002, and 6,740,655; U.S. Patent Application Nos. 20030187052, 20030187257, 20030144300, 20030130254, 20030186974, 20030220352, 20030134876, 20040048903, 20040023945, 20040044036, 20040106641, 20040097593, 20040242643, 20040192701, 20040224971, 20040220183, 20040180900, 20040171798, 20040167199, 20040146561, 20040152754, 20040229918, 20050192336, 20050267196, 20050049258, 20060014782, 20060004003, 20060019932, 20050267196, 20050222207, 20050222207, 20060009481; PCT Publication No. WO 92/079778; and Molnar-Kimber, K. L. et al. J. Immunol., 150:295A (1993), each of which is incorporated herein by reference.

Type V Phosphodiesterase Inhibitors

Type V PDE inhibitors that can be used in the methods, compositions, and kits of the invention include those described in U.S. Pat. Nos. 6,992,192, 6,984,641, 6,960,587, 6,943,166, 6,878,711, and 6,869,950, and U.S. Patent Application Nos. 20030144296, 20030171384, 20040029891, 20040038996, 20040186046, 20040259792, 20040087561, 20050054660, 20050042177, 20050245544, 20060009481, each of which is incorporated herein by reference.

Type VI Phosphodiesterase Inhibitors

Type VI PDE inhibitors that can be used in the methods, compositions, and kits of the invention include those described in U.S. Patent Application Nos. 20040259792, 20040248957, 20040242673, and 20040259880, each of which is incorporated herein by reference.

Type VII Phosphodiesterase Inhibitors

Type VII PDE inhibitors that can be used in the methods, compositions, and kits of the invention include those described in the following patents, patent application, and references: U.S. Pat. Nos. 6,838,559, 6,753,340, 6,617,357, and 6,852,720; U.S. Patent Application Nos. 20030186988, 20030162802, 20030191167, 20040214843, and 20060009481; PCT Publication WO 00/68230; and Martinez et al., J. Med. Chem. 43:683-689 (2000), each of which is incorporated herein by reference.

Non-Selective Phosphodiesterase Inhibitors

Non-selective PDE inhibitors that can be used in the methods, compositions, and kits of the invention include theophylline, papaverine, and ibudilast. Other PDE inhibitors that can be used in the methods, compositions, and kits of the invention are described in U.S. Pat. No. 6,953,774.

Tricyclic Compounds

Tricyclic compounds that can be used in the methods, compositions, and kits of the invention include amitriptyline, amoxapine, clomipramine, desipramine, dothiepin, doxepin, imipramine, lofepramine, maprotiline, mianserin, mirtazapine, nortriptyline, octriptyline, oxaprotiline, protriptyline, trimipramine, 10-(4-methylpiperazin-1-yl)pyrido(4,3-b)(1,4)benzothiazepine; 11-(4-methyl-1-piperazinyl)-5H-dibenzo(b,e)(1,4)diazepine; 5,10-dihydro-7-chloro-10-(2-(morpholino)ethyl)-11H-dibenzo(b,e)(1,4)diazepin-11-one; 2-(2-(7-hydroxy-4-dibenzo(b,f)(1,4)thiazepine-11-yl-1-piperazinyl)ethoxy)ethanol; 2-chloro-11-(4-methyl-1-piperazinyl)-5H-dibenzo(b,e)(1,4)diazepine; 4-(11H-dibenz(b,e)azepin-6-yl)piperazine; 8-chloro-11-(4-methyl-1-piperazinyl)-5H-dibenzo(b,e)(1,4)diazepin-2-ol; 8-chloro-11-(4-methyl-1-piperazinyl)-5H-dibenzo(b,e)(1,4)diazepine monohydrochloride; (Z)-2-butenedioate 5H-dibenzo(b,e)(1,4)diazepine; adinazolam; amineptine; amitriptylinoxide; butriptyline; clothiapine; clozapine; demexiptiline; 11-(4-methyl-1-piperazinyl)-dibenz(b,f)(1,4)oxazepine; 1′-(4-methyl-1-piperazinyl)-2-nitro-dibenz(b,f)(1,4)oxazepine; 2-chloro-11-(4-methyl-1-piperazinyl)-dibenz(b,f)(1,4)oxazepine monohydrochloride; dibenzepin; 11-(4-methyl-1-piperazinyl)-dibenzo(b,f)(1,4)thiazepine; dimetacrine; fluacizine; fluperlapine; imipramine N-oxide; iprindole; lofepramine; melitracen; metapramine; metiapine; metralindole; mianserin; mirtazapine; 8-chloro-6-(4-methyl-1-piperazinyl)-morphanthridine; N-acetylamoxapine; nomifensine; norclomipramine; norclozapine; noxiptilin; opipramol; oxaprotiline; perlapine; pizotyline; propizepine; quetiapine; quinupramine; tianeptine; tomoxetine; flupenthixol; clopenthixol; piflutixol; chlorprothixene; and thiothixene. Other tricyclic compounds are described, for example, in U.S. Pat. Nos. 2,554,736; 3,046,283; 3,310,553; 3,177,209; 3,205,264; 3,244,748; 3,271,451; 3,272,826; 3,282,942; 3,299,139; 3,312,689; 3,389,139; 3,399,201; 3,409,640; 3,419,547; 3,438,981; 3,454,554; 3,467,650; 3,505,321; 3,527,766; 3,534,041; 3,539,573; 3,574,852; 3,622,565; 3,637,660; 3,663,696; 3,758,528; 3,922,305; 3,963,778; 3,978,121; 3,981,917; 4,017,542; 4,017,621; 4,020,096; 4,045,560; 4,045,580; 4,048,223; 4,062,848; 4,088,647; 4,128,641; 4,148,919; 4,153,629; 4,224,321; 4,224,344; 4,250,094; 4,284,559; 4,333,935; 4,358,620; 4,548,933; 4,691,040; 4,879,288; 5,238,959; 5,266,570; 5,399,568; 5,464,840; 5,455,246; 5,512,575; 5,550,136; 5,574,173; 5,681,840; 5,688,805; 5,916,889; 6,545,057; and 6,600,065, and phenothiazine compounds that fit Formula (I) of U.S. patent application Ser. Nos. 10/617,424 or 60/504,310.

Standard recommended dosages for several tricyclic compounds are provided in Table 1, below. Other standard dosages are provided, e.g., in the Merck Manual of Diagnosis & Therapy (17th Ed. MH Beers et al., Merck & Co.) and Physicians' Desk Reference 2003 (57^{th} Ed. Medical Economics Staff et al., Medical Economics Co., 2002).

TABLE 1

| Compound | Standard Dose |
|---|---|
| Amoxapine | 200-300 mg/day |
| Nortriptyline | 75-150 mg/day |
| Desipramine | 100-200 mg/day |

Cotherapy

If desired, one or more additional agents may be administered in conjunction with the methods of the invention. Suitable agents include antibiotics (minocycline, penicillin, cephalosporin, tetracycline, oxytetracycline, chlortetracycline, metronidazole, chloramphenicol, streptomycin, neomycin, sulfonamides, phenolic compounds, quarternary ammonium compounds, doxycycline); antiseptics (e.g., chlorhexidine); nonsteroidal antiinflammatories (e.g., flurbiprofen, carprofen, diclofenac, fenbufen, fenclozic acid, fenoprofen, flufenamic acid, ibuprofen, indomethacin, indoprofen, ketoprofen, lonazolac, loxoprofen, meclofenamic acid, mefanamic acid, naproxen, proprionic acids, salicylic acids, sulindac, tolmetin, meloxicam, oxicams, piroxicam, tenoxicam, etodolac, and oxaprozin); tranexamic acid, allantoin; epsilon-aminocaproic acid; lysozyme; dihydrocholesterol; beta-glycyrrhetinic acid; platelet aggregation inhibitors (e.g., abciximab, aspirin, cilostazol, clopidogrel, eptifibatide, ticlopidine, or tirofiban); anticoagulants (e.g., dalteparin, danaparoid, enoxaparin, heparin, tinzaparin, or warfarin); antipyretics (e.g., acetaminophen); ticlopidine; clopidogrel; angiotensin converting enzyme inhibitors; beta blockers; pentoxifylline; cilostazol; estrogen replacement therapy; and lipid-lowering agents (e.g., cholestyramine, colestipol, nicotinic acid, gemfibrozil, probucol, ezetimibe, or statins such as atorvastatin, rosuvastatin, lovastatin simvastatin, pravastatin, cerivastatin, and fluvastatin). These agents may be administered concomitantly or within 14 days of the method of the invention. If desired, one or more of the foregoing agents is coformulated with one or more agents of the invention to form a single composition. Thus, in one embodiment, the invention features a tricyclic compound, one of the foregoing agents, and, optionally, a tetra-substituted pyrimidopyrimidine.

Dosages

The dosage of each compound of the claimed combinations depends on several factors, including: the administration method, the disease to be treated, the severity of the disease, whether the disease is to be treated or prevented, and the age, weight, and health of the person to be treated. Additionally, pharmacogenomic (the effect of genotype on the pharmacokinetic, pharmacodynamic or efficacy profile of a therapeutic) information about a particular patient may affect dosage used.

Continuous daily dosing with the combinations of the invention may not be required. A therapeutic regimen may require cycles, during which time a drug is not administered, or therapy may be provided on an as needed basis during periods of acute inflammation.

As described above, the compound in question may be administered orally in the form of tablets, capsules, elixirs or syrups, or rectally in the form of suppositories. Parenteral administration of a compound is suitably performed, for example, in the form of saline solutions or with the compound incorporated into liposomes. In cases where the compound in itself is not sufficiently soluble to be dissolved, a solubilizer such as ethanol can be applied.

Devices

One or more agents of the invention (e.g., amoxapine and/or dipyridamole) can be delivered to the periodontal pockets of a patient by way of a drug delivery device. Such devices are known in the art (see, e.g., U.S. Pat. Nos. 4,685,883; 5,262,164; 5,366,733; 5,447,725; 5,599,553; and 5,939,047).

The following examples are to illustrate the invention. They are not meant to limit the invention in any way.

### EXAMPLES

Study Protocol

We conducted an eight week blinded, randomized study with daily oral administration of the combination of amoxapine and dipyridamole or placebo, with weekly CRP and inflammatory cytokine measurements. The study population had severe periodontitis with at least 10 pockets of ≧5 mm in depth, with at least four pockets ≧6 mm. To qualify, 10% of the pockets must bleed on probing. The subject must otherwise have been in good general health.

During the study, the subjects were seen at the following study visits:
Screening visit (visit 1)
Day 1 (Baseline visit/visit 2)
Day 7±1 day (visit 3)
Day 14±1 day (visit 4)
Day 21±1 day (visit 5)
Day 35±1 day (visit 6)
Day 42×1 day (visit 7)
Day 43 (visit 8)
Day 49±1 day (visit 9)
Day 56±1 day (end of study visit/visit 10)
   Subjects were evaluated for study eligibility at the Screening visit, which was conducted within 14 days before the first dose of study drug. Treated subjects received scaling and root planing (SRP) treatment after 42 days (±1 day) on study drug, after pocket depth determination. All study subjects continued on study medication for an additional two weeks, after which serum CRP and inflammatory cytokine levels were determined on days 43, 49, and 56.

Subjects were randomized into treatment groups and received either amoxapine and dipyridamole at the doses indicated below or placebo tablets. In the treatment group there was one dose escalations after Day 14 as follows:
Days 1-14: Dose Level 1 (200 mg dipyridamole and 50 mg amoxapine)
Days 15-56: Dose Level 2 (200 mg dipyridamole and 100 mg amoxapine)

The drugs were blister packed for three dose levels as follows:

| | | |
|---|---|---|
| 8 am | 8 am | 1 pm |

Treatment group

| | | | |
|---|---|---|---|
| Day 1-14 | 50 mg | 100 mg | 100 mg |
| | amoxapine | dipyridamole | dipyridamole |
| Day 15-56 | 100 mg | 100 mg | 100 mg |
| | amoxapine | dipyridamole | dipyridamole |

Placebo

| | | | |
|---|---|---|---|
| Day 1-56 | Placebo | Placebo | Placebo |
| | (white) | (blue) | (white) |

The serum CRP level and inflammatory cytokine levels were determined using standard techniques. The results are shown in Tables 2-4.

TABLE 2
Summary of CRP by Study Visit
Post SRP Per-Protocol Population (b) (LOCF)

| | Placebo | | Amoxapine/Dipyridamole | |
|---|---|---|---|---|
| | | Percent Change | | Change from |
| | CRP | from Baseline | CRP | Baseline in |
| Visit | (mg/L) | in CRP | (mg/L) | CRP (mg/L) |

Day 42

| | | |
|---|---|---|
| N | 25 | 21 |
| Mean (Std Dev) | 3.36 (3.251) | 5.54 (5.033) |
| Median | 2.60 | 3.40 |
| Minimum, Maximum | 0.7, 15.2 | 0.6, 18.0 |

Day 43

| | | | | |
|---|---|---|---|---|
| N | 26 | 25 | 21 | 21 |
| Mean (Std Dev) | 19.07 (11.727) | 811.443 (1016.2456) | 20.04 (12.335) | 461.363 (470.6042) |
| Median | 16.60 | 613.953 | 17.70 | 241.000 |
| Minimum, Maximum | 4.6, 59.5 | 73.68, 5309.09 | 2.8, 42.2 | 35.85, 1909.52 |
| One Sided p-value (a) | | | | 0.0261 |

Day 49

| | | | | |
|---|---|---|---|---|
| N | 26 | 25 | 21 | 21 |
| Mean (Std Dev) | 4.80 (3.811) | 72.687 (107.6599) | 6.01 (6.832) | 40.723 (91.1396) |
| Median | 3.00 | 63.636 | 4.20 | 3.333 |
| Minimum, Maximum | 1.1, 18.1 | −61.02, 481.82 | 0.5, 33.4 | −75.65, 255.56 |

EOS/Day 56

| | | | | |
|---|---|---|---|---|
| N | 26 | 25 | 21 | 21 |
| Mean (Std Dev) | 4.02 (5.083) | 34.107 (96.4838) | 4.20 (3.360) | 2.927 (61.7295) |
| Median | 2.50 | −6.667 | 3.10 | −16.000 |
| Minimum, Maximum | 0.8, 25.2 | −73.53, 276.12 | 0.4, 12.3 | −87.83, 162.50 |

(a) P-value from Wilcoxon Rank Sum Test testing the null hypothesis there is no difference in percentage CRP change between the treatment groups from Day 42 to Day 43.
(b) Post SRP Per-Protocol Population is defined as Per-Protocol Population with at least 75% additional study drug complinace after Day 42.
Note:
SRP Baseline value collected at Day 42.

TABLE 3
Summary of TNF-Alpha by Study Visit
Post SRP Per-Protocol Population
(Patients Excluded Baseline TNF >2.5) (LOCF)

| | Placebo | | Amoxapine/Dipyridamole | |
|---|---|---|---|---|
| | | Change from | | Change from |
| | TNF-Alpha | Baseline in | TNF-Alpha | Baseline in |
| Visit | (pg/mL) | TNF-Alpha (pg/mL) | (pg/mL) | TNF-Alpha (pg/mL) |

SRP Baseline

| | | |
|---|---|---|
| N | 25 | 19 |
| Mean (Std Dev) | 0.9796 (0.52521) | 0.8356 (0.54915) |
| Median | 0.9110 | 0.6580 |
| Min, Max | 0.326, 2.408 | 0.120, 2.063 |

Day 43

| | | | | |
|---|---|---|---|---|
| N | 25 | 25 | 19 | 19 |
| LS mean (SE) | | 0.403 (0.1200) | | 0.186 (0.1400) |
| Mean (Std Dev) | 1.3730 (0.85984) | 0.3934 (0.54909) | 1.0335 (0.55544) | 0.1979 (0.64769) |
| Median | 1.2470 | 0.2860 | 0.9640 | 0.1350 |
| Min, Max | 0.373, 4.830 | −0.251, 2.422 | 0.119, 2.458 | −1.151, 2.318 |
| One Sided p-value (a) | | | | 0.1214 |
| Difference (two sided 95% CI) | | | | 0.200 (−0.1500, 0.5900) |

Day 49

| | | | | |
|---|---|---|---|---|
| N | 25 | 25 | 19 | 19 |
| Mean (Std Dev) | 0.9374 (0.61457) | −0.0421 (0.28630) | 0.7246 (0.49274) | −0.1110 (0.46019) |
| Median | 0.6640 | −0.0290 | 0.6060 | −0.0080 |
| Min, Max | 0.347, 2.800 | −0.747, 0.728 | 0.112, 2.258 | −1.388, 0.602 |

EOS/Day 56

| | | | | |
|---|---|---|---|---|
| N | 25 | 25 | 19 | 19 |
| Mean (Std Dev) | 1.1843 (1.29473) | 0.2048 (1.29220) | 0.6992 (0.34822) | −0.1364 (0.53249) |
| Median | 0.8380 | −0.0110 | 0.7220 | −0.0140 |
| Min, Max | 0.320, 6.767 | −0.630, 6.148 | 0.120, 1.230 | −1.641, 0.692 |

(a) P-value from ANCOVA adjusted for Post SRP baseline value testing the null hypothesis there is no difference in mean TNF-Alpha change between the treatment groups at Day 43.
Note1:
LOCF is done by Investigator.
Note2:
Baseline used Post SRP baseline which was collected at Day 42.

TABLE 4
Summary of IL-6 by Study Visit
Post SRP Per-Protocol Population (LOCF)

| | Placebo | | Amoxapine/Dipyridamole | |
|---|---|---|---|---|
| | | Change from | | Change from |
| | IL-6 | Baseline in | IL-6 | Baseline in |
| Visit | (pg/mL) | IL-6 (pg/mL) | (pg/mL) | IL-6 (pg/mL) |

SRP Baseline

| | | |
|---|---|---|
| N | 26 | 21 |
| Mean (Std Dev) | 1.4448 (1.04132) | 1.8113 (1.60985) |
| Median | 1.2500 | 1.1340 |
| Min, Max | 0.101, 4.983 | 0.452, 6.883 |

Day 43

| | | | | |
|---|---|---|---|---|
| N | 26 | 26 | 21 | 21 |
| LS mean (SE) | | 1.703 (0.3700) | 1.450 (0.4100) | |
| Mean (Std Dev) | 3.1928 (1.99488) | 1.7479 (2.10655) | 3.2062 (2.20320) | 1.3949 (1.58021) |
| Median | 2.6505 | 1.2040 | 2.8660 | 1.2310 |
| Min, Max | 0.681, 8.978 | −0.848, 8.746 | 0.186, 9.981 | −2.418, 5.272 |
| One Sided p-value (a) | | | | 0.3259 |
| Difference (two sided 95% CI) | | | | 0.300 (−0.8700, 1.3700) |

Day 49

| | | | | |
|---|---|---|---|---|
| N | 26 | 26 | 21 | 21 |
| Mean (Std Dev) | 1.2334 (0.83232) | −0.2115 (0.66662) | 2.5544 (6.00664) | 0.7431 (4.88289) |
| Median | 0.9860 | −0.0740 | 1.2890 | −0.0890 |
| Min, Max | 0.553, 4.501 | −1.650, 0.777 | 0.095, 28.459 | −3.201, 21.576 |

EOS/Day 56

| | | | | |
|---|---|---|---|---|
| N | 26 | 26 | 21 | 21 |
| Mean (Std Dev) | 1.4943 (1.28413) | 0.0495 (1.08615) | 1.1725 (0.88955) | −0.6388 (1.26151) |
| Median | 1.1530 | 0.1000 | 1.0340 | −0.3100 |
| Min, Max | 0.090, 5.199 | −2.114, 2.734 | 0.025, 3.583 | −3.600, 0.807 |

(a) P-value from ANCOVA adjusted for Post SRP baseline value testing the null hypothesis there is no difference in mean IL-6 change between the treatment groups at Day 43.
Note1:
LOCF is done by Investigator.
Note2:
Baseline used Post SRP baseline which was collected at Day 42.

### OTHER EMBODIMENTS

Various modifications and variations of the described methods and compositions of the invention will be apparent to those skilled in the art without departing from the scope and spirit of the invention. Although the invention has been described in connection with specific desired embodiments, it should be understood that the invention as claimed should not be unduly limited to such specific embodiments. Indeed, various modifications of the described modes for carrying out the invention that are obvious to those skilled in the fields of medicine, immunology, pharmacology, endocrinology, or related fields are intended to be within the scope of the invention.

All publications mentioned in this specification are herein incorporated by reference to the same extent as if each independent publication was specifically and individually incorporated by reference.

What is claimed is:

1. A method for reducing the serum C-reactive protein (CRP) level in a patient in need thereof, said method comprising administering to said patient (i) a tricyclic compound; and (ii) a tetra-substituted pyrimidopyrimidine, wherein said tricyclic compound and tetra-substituted pyrimidopyrimidine are each administered in amounts and for a duration that together are sufficient to reduce the serum CRP level in said patient.

2. A method for treating a disease or condition associated with an increased serum CRP level in a patient in need thereof, said method comprising administering to said patient (i) a tricyclic compound; and (ii) a tetra-substituted pyrimidopyrimidine, wherein said tricyclic compound and tetra-substituted pyrimidopyrimidine are each administered in amounts and for a duration that together are sufficient to reduce the serum CRP level in said patient.

3. The method of claim 2, wherein said disease or condition associated with an increased serum CRP level is selected from the group consisting of cardiovascular disease, atherosclerosis, hypertension, giant cell arteritis, Kawasaki disease, familial cold urticaria, angina pectoris, vascular insults, end-stage renal disease, colon cancer, lymphoma, sarcoma, pancreatitis, and pancreatic cancer.

4. The method of claim 1, wherein said tricyclic compound is selected from the group consisting of amitriptyline, amoxapine, clomipramine, dothiepin, doxepin, desipramine, imipramine, lofepramine, loxapine, maprotiline, mianserin, mirtazapine, oxaprotiline, nortriptyline, octriptyline, protriptyline, and trimipramine.

5. The method of claim 1, wherein said tetra-substituted pyrimidopyrimidine is selected from the group consisting of 2,6-disubstituted 4,8-dibenzylaminopyrimido[5,4-d]pyrimidines; mopidamole; dipyridamole monoacetate; 1-((2,7-bis(2-methyl-4-morpholinyl)-6-phenyl-4-pteridinyl)(2-hydroxyethyl)amino)-2-propanol; TX-3301; 2,6-di-(2,2-dimethyl-1,3-dioxolan-4-yl)-methoxy-4,8-di-piperidinopyrimidopyrimidine; 2,6-bis-(2,3-dimethyoxypropoxy)-4,8-di-piperidinopyrimidopyrimidine; 2,6-bis[N,N-di(2-methoxy)ethyl]-4,6-di-piperidinopyrimidopyrimidine; and 2,6-bis(diethanolamino)-4,8-di-4-methoxybenzylaminopyrimidopyrimidine.

6. The method of claim 5, wherein said tricyclic compound is amoxapine and said tetra-substituted pyrimidopyrimidine is dipyridamole.

7. The method of claim 1, further comprising administering to said patient a third agent selected from the group consisting of antibiotics; antiseptics; nonsteroidal antiinflammatories; tranexamic acid; allantoin; epsilon-aminocaproic acid; lysozyme; dihydrocholesterol; and beta-glycyrrhetinic acid.

8. The method of claim 1, wherein said tricyclic compound and tetra-substituted pyrimidopyrimidine are formulated in a single composition.

9. The method of claim 8, wherein said composition is formulated for oral administration.

10. The method of claim 8, wherein said composition is formulated for systemic administration.

11. The method of claim 1, wherein said tricyclic compound and tetra-substituted pyrimidopyrimidine are administered simultaneously or within 14 days of each other.

12. The method of claim 1, wherein said tricyclic compound or said tetra-substituted pyrimidopyrimidine is present in said composition in a low dosage.

13. A method for treating periodontal disease, said method comprising administering to a patient (i) a tricyclic compound; and (ii) a tetra-substituted pyrimidopyrimidine, wherein said tricyclic compound and tetra-substituted pyrimidopyrimidine are each administered in amounts and for a duration that together are sufficient to treat periodontal disease.

14. The method of claim 13, wherein the periodontal disease is periodontitis or gingivitis.

15. A device for administering drugs to the periodontal pockets of a patient having periodontal disease, said device comprising a tricyclic compound and a tetra-substituted pyrimidopyrimidine capable of being released into the periodontal pockets of said patient.

16. The device of claim 15, wherein said tricyclic compound is selected from the group consisting of amitriptyline, amoxapine, clomipramine, dothiepin, doxepin, desipramine, imipramine, lofepramine, loxapine, maprotiline, mianserin, mirtazapine, oxaprotiline, nortriptyline, octriptyline, protriptyline, and trimipramine.

17. The device of claim 16, wherein said tetra-substituted pyrimidopyrimidine is selected from the group consisting of 2,6-disubstituted 4,8-dibenzylaminopyrimido[5,4-d]pyrimidines; mopidamole; dipyridamole monoacetate; 1-((2,7-bis(2-methyl-4-morpholinyl)-6-phenyl-4-pteridinyl)(2-hydroxyethyl)amino)-2-propanol; TX-3301; 2,6-di-(2,2-dimethyl-1,3-dioxolan-4-yl)-methoxy-4,8-di-piperidinopyrimidopyrimidine; 2,6-bis-(2,3-dimethyoxypropoxy)-4,8-di-piperidinopyrimidopyrimidine; 2,6-bis[N,N-di(2-methoxy)ethyl]-4,6-di-piperidinopyrimidopyrimidine; and 2,6-bis(diethanolamino)-4,8-di-4-methoxybenzylaminopyrimidopyrimidine.

18. The device of claim 17, wherein said tricyclic compound is amoxapine and said tetra-substituted pyrimidopyrimidine is dipyridamole.

19. The device of claim 15, further comprising a third agent selected from the group consisting of antibiotics; antiseptics; nonsteriodal antiinflammatories; tranexamic acid; allantoin; epsilon-aminocaproic acid; lysozyme; dihydrocholesterol; and beta-glycyrrhetinic acid.

20. A kit comprising:
(i) a tricyclic compound;
(ii) a tetra-substituted pyrimidopyrimidine; and
(iii) instructions for administering said tricyclic compound and said tetra-substituted pyrimidopyrimidine to a patient having or at risk of having (a) periodontal disease, or (b) an increased serum CRP level.

21. A kit comprising:
(i) a tricyclic compound or a tetra-substituted pyrimidopyrimidine; and
(ii) instructions for administering said tricyclic compound and said tetra-substituted pyrimidopyrimidine to a patient having or at risk of having (a) periodontal disease, or (b) an increased serum CRP level.

22. A kit comprising:
(i) a composition comprising a tricyclic compound and a tetra-substituted pyrimidopyrimidine; and
(ii) instructions for administering said composition to a patient having or at risk of having (a) periodontal disease, or (b) an increased serum CRP level.
